(19) United States
(12) Patent Application Publication
(10) Pub. No.: US 2025/0235717 A1
(43) Pub. Date: Jul. 24, 2025

(54) LIGHT THERAPY LOUNGER AND METHOD OF MANUFACTURING THEREOF

(71) Applicant: ENERGY LOUNGER LLC, MIAMI, FL (US)

(72) Inventors: ALAIN DIJKSTRA, Amstelveen (NL); Barry Warrington, Miami, FL (US)

(73) Assignee: ENERGY LOUNGER LLC, MIAMI, FL (US)

(21) Appl. No.: 18/415,714

(22) Filed: Jan. 18, 2024

(51) Int. Cl. A61N 5/067; A61N 5/06; G16H 40/63

(52) U.S. Cl. CPC A61N 5/067; G16H 40/63; A61N 2005/0626; A61N 2005/0632; A61N 2005/0638; A61N 2005/0652; A61N 2005/0653; A61N 2005/0659; A61N 2005/0661

(57) ABSTRACT

Embodiments of the present invention disclose a light therapy lounger. The light therapy lounger includes a frame including a peripheral wall and an inward protrusion extending internally from an inner surface of the peripheral wall. Further, the light therapy lounger includes a plurality of irradiation boards located on the inward protrusion and including a plurality of irradiation sources. Also, the light therapy lounger includes an upper cover made up of a diaphanous material, the upper cover configured to be located above the plurality of irradiation boards. Each irradiation board includes one or more pressure sensors and several irradiation sources electrically coupled to a first Printed Circuit Board (PCB), a heat sink thermally coupled to the first PCB and located under the first PCB, and a plurality of cooling fans electrically coupled to the first PCB and/or a second PCB, and located under the heat sink.

### TECHNICAL FIELD

The present invention relates generally to recreational and therapeutic irradiation apparatus. More specifically, the present invention relates to reclining furniture to be used as a medium for providing the irradiation for therapeutic and recreational applications.

### BACKGROUND ART

Sauna beds and pods, designed for both recreational and therapeutic purposes, are readily accessible in the market. Substantial research has underscored the physiological advantages associated with these products. Nonetheless, sauna beds, pods, and their variations are relatively constrained in their functionalities. The majority of these products rely on far-infrared radiation, positioned at the far end of the electromagnetic spectrum, to induce body heating. Light Emitting Diodes (LEDs) are frequently employed as the primary source for generating far-infrared radiation for this purpose. However, these products fall short in delivering finely tuned and precisely targeted benefits, even though certain cases have documented residual advantages such as skin rejuvenation and pain relief. Consequently, several solutions have been proposed within the field of sauna beds and pods to address these limitations.

U.S. Pat. No. 7,503,926B2 reveals a personal therapy or sauna enclosure featuring a base, offering a space for reclining. A cover is placed over this base, enveloping the individual's body, leaving the head outside the cover. The bed hovers above massage showerheads that deliver upward water massage to the occupant's back. Infrared heaters are integrated into the cover, with showerheads ensuring both comfort and bathing for the person lying on the bed. Additionally, light support panels incorporate multi-colored LED lights, projecting light toward the user's head resting on the pad or mattress. The lights are viewable directly and produce reflected light. However, this disclosed enclosure is constrained by its intricate construction and the resulting manufacturing expenses. Furthermore, several of the described mechanisms, such as the water impingement, demand a relatively high power consumption.

U.S. Pat. No. 9,005,101B1 introduces a system and technique for administering targeted therapy to different body areas based on detected biological indicators. The patent document describes a substrate equipped with a multitude of pressure and thermal sensors, ensuring precise positioning of a person lying on it, such as a bed or mattress. Additionally, it incorporates various sensors capable of monitoring diverse biological parameters, including temperature, acceleration, moisture, resistance, stress levels, heart rate, respiration rate, brain waves, blood flow rate, metabolic activity, and blood oxygenation, among others. Using the feedback from these biological indicators, the therapy devices linked to the substrate are programmed to provide the desired treatments. Nonetheless, this disclosed system grapples with limitations due to the intricate nature of its control architecture, offering limited insight into the specific workings of this control architecture. Moreover, the document does not provide adequate details regarding the structural support necessary for the control architecture or the packaging of multiple electronic components for user-friendly convenience.

US Pat. App. No. 2018/0110960 A1 unveils a system and methodology for alleviating stress and enhancing sleep by regulating the temperature of a mattress pad's surface through a fluid-based system. This system encompasses an array of bodily sensors, including a respiration sensor, electrooculography sensor, heart rate sensor, bodyweight sensor, electromyography sensor, brain wave sensor, temperature sensor, analyte sensor, pulse oximeter sensor, blood pressure sensor, electrodermal activity sensor, and body weight sensor. These sensors monitor various parameters of an individual lying on the bed, facilitating the prediction of values essential for the stress reduction and sleep enhancement system. In addition to this, the system incorporates a red or infrared light-emitting device for therapeutic purposes. However, it's important to note that the mattress's heating is accomplished through the circulation of a working fluid, which necessitates relatively cumbersome and intricate hydraulic or pneumatic systems, resulting in significantly elevated electricity consumption.

The aforementioned solutions, although promising, either lack detailed construction information or involve complex and cost-prohibitive designs. As a result, there is a demand in the industry for a solution that overcomes the aforementioned deficiencies.

### OBJECTS OF THE INVENTION

Some of the objects of the invention are as follows:

An object of the present invention is to provide a lounger capable of delivering light therapy (or Photo-Biomodulation) using irradiation sources provided inside of a frame of lounger.

Another object of the present invention is to provide a lounger that is capable of delivering targeted light therapy based on identification of specific body portions, muscle groups, and blood vessels.

Another object of the present invention is to provide a lounger that is capable of absorbing and dissipating the heat generated by the irradiation sources during usage of the lounger.

Another object of the present invention is to provide a lounger which can serve an additional purpose of wirelessly charging an electronic device placed in a predesigned cavity.

Another object of the present invention is to provide a lounger that is provided with several sensors enabling the lounger to scan an immediate environment of the lounger and automatically activate or deactivate depending upon an approach of a user towards or away from the lounger, respectively.

Another object of the present invention is to provide a lounger in which the sensors and control architecture of the lounger further enables the lounger to identify a demographic and/or species of the user and modify irradiation characteristics of the irradiation sources based upon the identified demographic and/or the species.

Another object of the present invention is to provide a lounger in which the control architecture of the lounger can communicate with a user computing device and receive control signals for modifying several operating parameters of the lounger.

It is also an objective of the present invention to provide a lounger in which the control architecture is capable of deploying machine learning algorithms and artificial intelligence to perform several tasks including the targeted irradiation, the automatic activation and deactivation, and the identification of the demographic and/or the species of the user, and modification of the irradiation characteristics of the irradiation sources.

### SUMMARY OF THE INVENTION

According to a first aspect of the present invention, there is provided a light therapy lounger. The light therapy lounger includes a frame including a peripheral wall and an inward protrusion extending internally from an inner surface of the peripheral wall, the inward protrusion disposed at a predetermined depth from an upper surface of the peripheral wall. Further, the light therapy lounger includes a plurality of irradiation boards including a plurality of irradiation sources, the plurality of irradiation boards configured to be located on the inward protrusion. The light therapy lounger also includes an upper cover made up of a diaphanous material, the upper cover configured to be located above the plurality of irradiation boards. Each irradiation board of the plurality of irradiation boards includes one or more pressure sensors and several irradiation sources electrically coupled to a first Printed Circuit Board (PCB), a heat sink thermally coupled to the first PCB and located under the first PCB, and a plurality of cooling fans electrically coupled to the first PCB and/or a second PCB, and located under the heat sink.

In one embodiment of the invention, the inward protrusion includes a first protrusion portion parallel to a locating surface for locating the frame, a second protrusion portion at a first predetermined angle to the first protrusion portion and extending rearwardly and downwardly, and a third protrusion portion at a second predetermined angle to the second protrusion portion and extending rearwardly and upwardly, the plurality of irradiation boards includes at least a first irradiation board configured to be located on the first protrusion portion, at least a second irradiation board configured to be located on the second protrusion portion, and at least a third irradiation board configured to be located on the third protrusion portion, and the upper cover includes a first cover portion parallel to the first protrusion portion, a second cover portion parallel to the second protrusion portion, and a third cover portion parallel to the third protrusion portion.

In one embodiment of the invention, the plurality of irradiation sources is selected from a group consisting of Light Emitting Diodes (LEDs) and lasers.

In one embodiment of the invention, the light therapy lounger further includes one or more exhaust vents located in the frame for dissipating heated air generated by the plurality of cooling fans.

In one embodiment of the invention, the light therapy lounger further includes a wireless charging pod provided with a transmitter induction coil, the wireless charging pod configured to receive an electronic device including a receiver induction coil, the receiver induction coil configured to generate an Electro-motive force (EMF) when brought within a time-varying magnetic field generated by the transmitter induction coil.

In one embodiment of the invention, the light therapy lounger further includes a user interface configured to receive a control input signal to modify irradiation characteristics of the plurality of irradiation sources.

In one embodiment of the invention, the frame further includes a lower cover made up of aluminum material.

In one embodiment of the invention, the light therapy lounger further includes a plurality of sensors, the one or more pressure sensors representing a subset of the plurality of sensors, a processor, and a memory unit, the memory unit including machine-readable instructions. The machine-readable instructions when executed by the processor, enable the processor to receive input data from the plurality of sensors, the input data indicative of presence of a user within a predefined 3-Dimensional space around the frame, determine a location of the user using the input data, and activate one or more irradiation sources of the plurality of irradiation sources directed towards the location of the user.

In one embodiment of the invention, the processor is further enabled to determine a demographic and/or a species to which the user belongs, and modify irradiation characteristics of the one or more irradiation sources based on the determined demographic and/or species.

In one embodiment of the invention, the processor is further enabled to identify a location of one or more of a predetermined body portion, a predetermined muscle group, and a predetermined group of blood vessels, and activate one or more irradiation sources of the plurality of irradiation sources, the activated one or more irradiation sources directed towards the location of the one or more of the identified predetermined body portion, the predetermined muscle group, and the predetermined group of blood vessels.

In one embodiment of the invention, the light therapy lounger further includes a communication interface configured to receive a control input signal, from a user computing device, the processor further enabled to modify irradiation characteristics of the plurality of irradiation sources in response to the receipt of the control input signal.

In one embodiment of the invention, the machine-readable instructions included in the memory unit correspond to implementation of Artificial Intelligence (AI) developed through Machine Learning and/or Deep Learning algorithms trained on historical training data.

According to a second aspect of the present invention, there is provided a light therapy lounger. The light therapy lounger includes a frame including a peripheral wall and an inward protrusion extending internally from an inner surface of the peripheral wall, the inward protrusion disposed at a predetermined depth from an upper surface of the peripheral wall. Further, the light therapy lounger includes a plurality of irradiation boards including a plurality of irradiation sources, the plurality of boards configured to be located on the inward protrusion, wherein each irradiation board of the plurality of irradiation boards includes one or more pressure sensors and several irradiation sources electrically coupled to a first Printed Circuit Board (PCB), a heat sink thermally coupled to the first PCB and located under the first PCB, and a plurality of cooling fans electrically coupled to the first PCB and/or a second PCB, and located under the heat sink. The light therapy lounger further includes an upper cover made up of a diaphanous material, the upper cover configured to be located above the plurality of irradiation boards. Furthermore, the light therapy lounger includes a plurality of sensors including a plurality of proximity sensors and a plurality of pressure sensors. The light therapy lounger further includes a memory unit, the memory unit including machine-readable instructions that correspond to implementation of Artificial Intelligence (AI) developed through Machine Learning and/or Deep Learning algorithms trained on historical training data. The light therapy lounger also includes a processor operably connected to the memory unit. The machine-readable instructions when executed by the processor, enable the processor to perform one or more of receive input data from the plurality of sensors, the input data indicative of presence of a user within a predefined 3-Dimensional space around the frame, determine a location of the user using the input data, determine a demographic and/or a species to which the user belongs, identify a location of one or more of a predetermined body portion, a predetermined muscle group, and a predetermined group of blood vessels, activate one or more of irradiation sources of the plurality of irradiation sources directed towards the location of the one or more of the identified predetermined body portion, the predetermined muscle group, and the predetermined set of blood vessels, and modify irradiation characteristics of the activated one or more irradiation sources based on the determined demographic and/or the species.

In one embodiment of the invention, the light therapy lounger further includes a communication interface configured to receive a control input signal, from a user computing device, the processor further enabled to modify irradiation characteristics of the plurality of irradiation sources in response to the receipt of the control input signal.

In one embodiment of the invention, the inward protrusion includes a first protrusion portion parallel to a locating surface for locating the frame, a second protrusion portion at a first predetermined angle to the first protrusion portion and extending downwardly, and a third protrusion portion at a second predetermined angle to the second protrusion portion and extending upwardly, the plurality of irradiation boards includes at least a first irradiation board configured to be located on the first protrusion portion, a second irradiation board configured to be located on the second protrusion portion, and a third irradiation board configured to be located on the third protrusion portion, and the upper cover includes a first cover portion parallel to the first protrusion portion, a second cover portion at the first predetermined angle to the first cover portion and extending downwardly, and a third cover portion at the second predetermined angle to the second cover portion and extending upwardly.

According to a third aspect of the present invention, there is provided a method of manufacturing a light therapy lounger. The method includes fabricating a frame including a peripheral wall and an inward protrusion extending internally from an inner surface of the peripheral wall, the inward protrusion disposed at a predetermined depth from an upper surface of the peripheral wall. The method further includes fabricating a plurality of irradiation boards including a plurality of irradiation sources. Furthermore, the method includes fabricating an upper cover made up of a diaphanous material. The method also includes assembling the frame, the plurality of irradiation boards and the upper cover, such that, the plurality of irradiation boards are located on the inward protrusion and the upper cover is located above the plurality of irradiation boards. Each irradiation board of the plurality of irradiation boards includes one or more pressure sensors and several irradiation sources electrically coupled to a first Printed Circuit Board (PCB), a heat sink thermally coupled to the first PCB and located under the first PCB, and a plurality of cooling fans electrically coupled to the first PCB and/or a second PCB and located under the heat sink.

In one embodiment of the invention, the inward protrusion includes a first protrusion portion parallel to a locating surface for locating the frame, a second protrusion portion at a first predetermined angle to the first protrusion portion and extending rearwardly and downwardly, and a third protrusion portion at a second predetermined angle to the second protrusion portion and extending rearwardly and upwardly, the plurality of irradiation boards includes at least a first irradiation board configured to be located on the first protrusion portion, at least a second irradiation board configured to be located on the second protrusion portion, and at least a third irradiation board configured to be located on the third protrusion portion, and the upper cover includes a first cover portion parallel to the first protrusion portion, a second cover portion parallel to the second protrusion portion, and a third cover portion parallel to the third protrusion portion.

In one embodiment of the invention, the method further includes providing one or more exhaust vents located in the frame for dissipating heated air generated by the plurality of cooling fans.

In one embodiment of the invention, the method further includes providing a user interface configured to receive a control input signal to modify irradiation characteristics of the plurality of irradiation sources.

In one embodiment of the invention, the method further includes providing, in the frame, a wireless charging pod provided with a transmitter induction coil, the wireless charging pod configured to receive an electronic device including a receiver induction coil, the receiver induction coil configured to generate an Electro-motive force (EMF) when brought within a time-varying magnetic field generated by the transmitter induction coil.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, a “diaphanous material” is a material that allows at least a portion of one or more forms (such as Infrared, Ultraviolet, X-Rays, Visible Light, Microwaves, Radio Waves, etc.) of electromagnetic radiation to pass through them. The diaphanous materials can be transparent (allowing the one or more forms of the electromagnetic radiation to pass through with minimal scattering) or translucent (allowing the one or more forms of the electromagnetic radiation to pass through with appreciable diffusion or scattering). Diaphanous materials can be dense, like glass, or have an open structure, like wire mesh or a woven fabric.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Bluc) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a top perspective view of a light therapy lounger, in accordance with an embodiment of the present invention;

FIG. 1B illustrates a bottom perspective view of the light therapy lounger of FIG. 1A;

FIG. 1C illustrates a right side view of the light therapy lounger of FIG. 1A;

FIG. 1D illustrates a left side view of the light therapy lounger of FIG. 1A;

FIG. 1E illustrates an exploded view of the light therapy lounger of FIG. 1A;

FIG. 1F illustrates a detailed view of a portion B of the light therapy lounger of FIG. 1A, and a sectional view of the light therapy lounger, the section taken along a plane A-A depicted in FIGS. 1C and 1D;

FIG. 2A illustrates a perspective view of an irradiation board of the light therapy lounger, in accordance with an embodiment of the present invention;

FIG. 2B illustrates an exploded view of the irradiation board of FIG. 2A;

FIG. 3 illustrates an environment depicting two irradiation boards in communication with a user computing device, through a communication network, in accordance with an embodiment of the present invention;

FIG. 4 illustrates a user approaching a detection field in vicinity of the light therapy lounger, in accordance with an embodiment of the present invention;

FIG. 5 illustrates a use case scenario of the light therapy lounger, with the user lying on the light therapy lounger, in accordance with an embodiment of the present invention; and

FIG. 6 illustrates a method of manufacturing the light therapy lounger, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a light therapy lounger. The light therapy lounger has been provided with a frame and an upper cover located upon the frame. The frame can be made up of combinations of polymeric and metallic materials. Some examples of polymeric materials with relatively higher strength-to-weight ratios include Polyethylene Terephthalate, Fiber-Reinforced Polymer (FRP) with Unsaturated Polyester (UP) used as a resinous material, Vinyl Ester, Epoxy, Polyurethane, etc. The metallic materials may include wrought iron. alloys of iron, aluminum, etc. Moreover, the upper cover is made up of a diaphanous material. Some of the examples of diaphanous materials include Polycarbonate, PMMA or Acrylic, Polyethylene Terephthalate (PET), Amorphous Co-polyester (PETG), Polyvinyl Chloride (PVC), Liquid Silicone Rubber (LSR), Cyclic Olefin Copolymers, Polyethylene (PE), Polystyrene (PS), Thermoplastic polyurethanes (TPU), Polyvinyl butyral (PVB), Co-polymer ethylene vinyl acetate (EVA). Several irradiation boards have been located in between the frame and the upper cover. Each one of the several irradiation boards has been provided with several irradiation sources, such as Light Emitting Diodes (LEDs) or lasers, in a manner, that the light-emitting portion of the irradiation sources is directed upwards towards the upper cover. During operation, at least a part of all the irradiation sources combined emit light of predefined wavelengths towards the upper cover. Since the upper cover is made from a diaphanous material, a user lying on the upper cover receives the emitted light for therapeutic and recreational purposes.

To dissipate the heat generated by the irradiation sources each one of the irradiation boards has been provided with a heat sink and several cooling fans. The heat sinks may be made up of aluminum and provided with several fins on the outer surface of the heat sink to increase the surface area of the heat sink for enhanced heat dissipation. Moreover, additional vents and cooling fans have also been provided in the frame to prevent heated air released by the cooling fans of the irradiation boards from accumulating inside the frame. The frame is additionally provided with ground-engaging members such as wheels to make the light therapy lounger easy to transport from one place to another. In addition, suction cups may be provided at a lower portion of the frame to adhere the light therapy lounger to a locating surface on which the light therapy lounger has been located. Additional structural features such as a user interface for the monitoring and control of the operation of the light therapy lounger and a wireless charging pod for charging an electronic device have been provided as coupled to the frame.

Furthermore, to at least partially automate the operation of the light therapy lounger, several sensors such as capacitive antennae acting as pressure sensors, and proximity sensors such as hall effect sensors and optical sensors have also been provided in the light therapy lounger. The optical sensors would allow an on-board control module to detect the presence or an approach of the user towards the light therapy lounger, in addition to a demographic (a child, an adult, a man, a woman, etc.) of the user, a species of the user (such as a person or a pet cat or a pet dog), physiological characteristics (such as height, estimated weight, skin type, complexion, etc.), a visible medical condition (such as inflammation, lacerations of the skin, microbial infection such as ringworm, etc.), etc. In addition, pressure sensors would allow the control module to identify body portions, muscle groups, groups of blood vessels, etc.

The signals received from the several sensors would allow the control module to further modify the operational characteristics of the irradiation sources that are best suited to provide an enhanced therapeutic and recreational benefit to the user. In that regard, the control module may be pre-trained to identify information such as the physiological characteristics, medical condition, and an optimal treatment schedule based on historical training data to a Machine Learning (ML) algorithm, thereby providing the light therapy lounger with Artificial Intelligence (AI) capabilities. Also, the control module is configured to receive control signals from a user computing device and factor in control signals received from the user computing device while determining the operational characteristics of the irradiation sources.

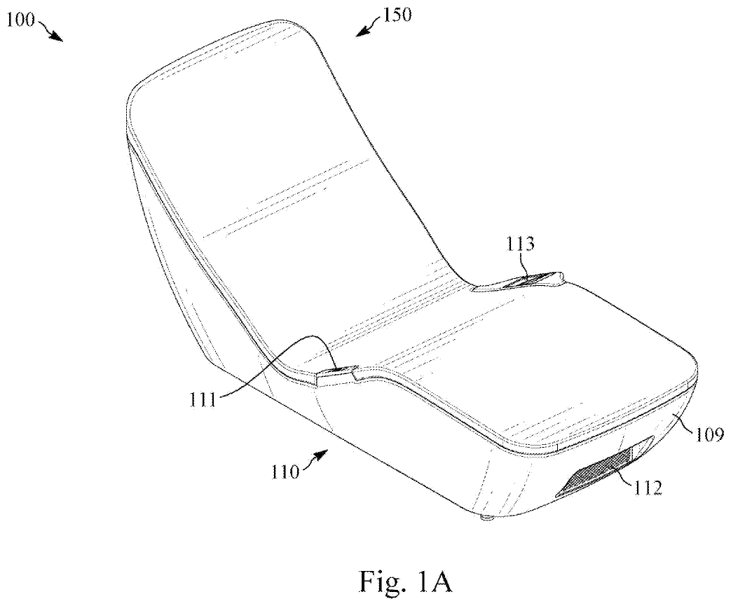

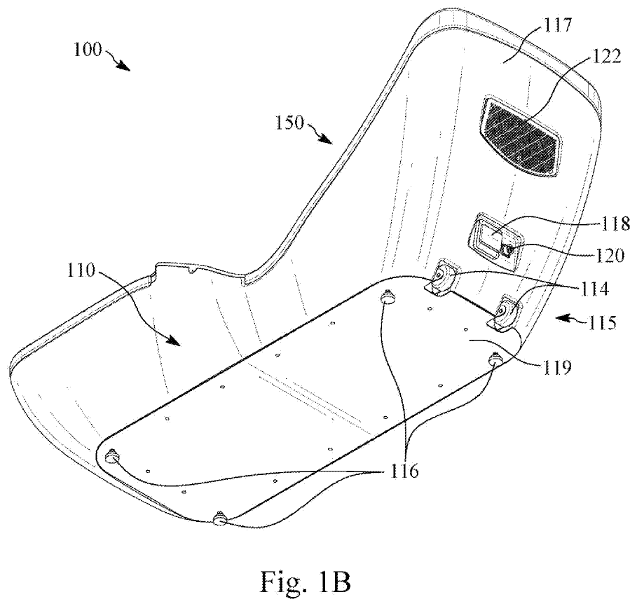

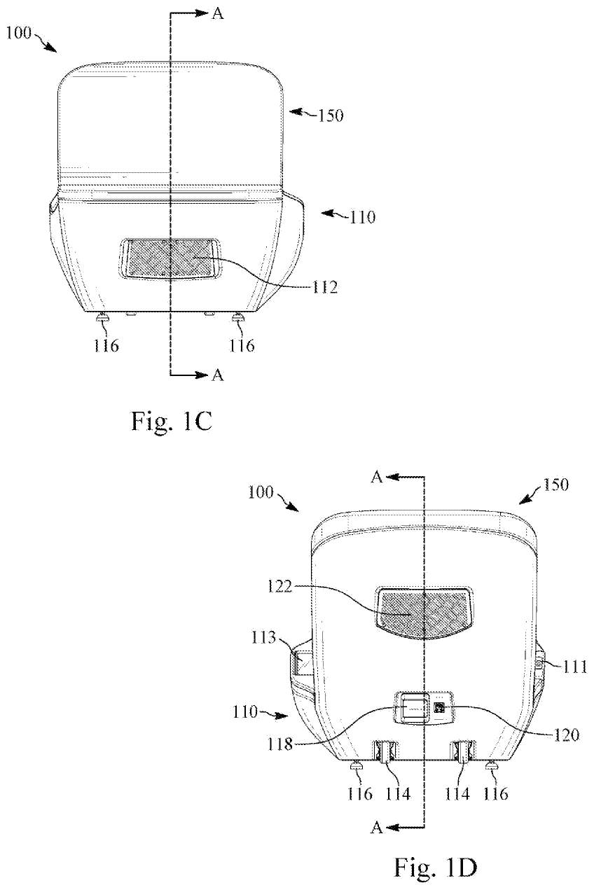

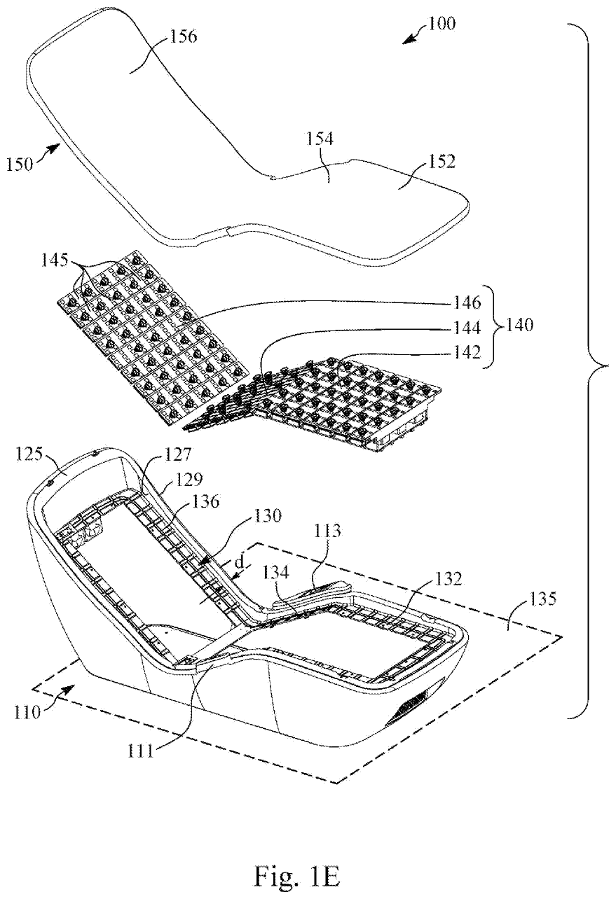

Several embodiments of the present invention will now be described in detail with references to FIGS. 1A-6.

FIG. 1A illustrates a top perspective view of a light therapy lounger 100 (hereinafter also referred to as “the lounger 100”), in accordance with an embodiment of the present invention. FIG. 1B illustrates a bottom perspective view of the lounger 100 of FIG. 1A. FIG. 1C illustrates a right-side view of the lounger 100 of FIG. 1A. FIG. 1D illustrates a left-side view of the lounger 100 of FIG. 1A. FIG. 1E illustrates an exploded view of the lounger 100 of FIG. 1A. Also, FIG. 1F illustrates a detailed view of portion B of the lounger 100 of FIG. 1A, and a sectional view of the lounger 100, the section taken along plane A-A depicted in FIGS. 1C and 1D. The following discussion collectively refers to FIGS. 1A-1F.

The lounger 100 includes a frame 110 and an upper cover 150. The frame 110 may be made from a polymer material, a metallic material, a composite material, or combinations thereof. The frame 110 encloses a cavity in which several structural, electrical, and electronic components of the lounger 100 have been installed. The upper cover 150 is envisaged to be made up of a diaphanous material. Several examples of diaphanous materials have been listed in the aforementioned discussion. The frame 100 also includes one or more exhaust vents, such as a first exhaust vent 112 provided in a front portion 109 of the frame 110, to allow heated air to exit from within the frame 110 during the operation of the lounger 100.

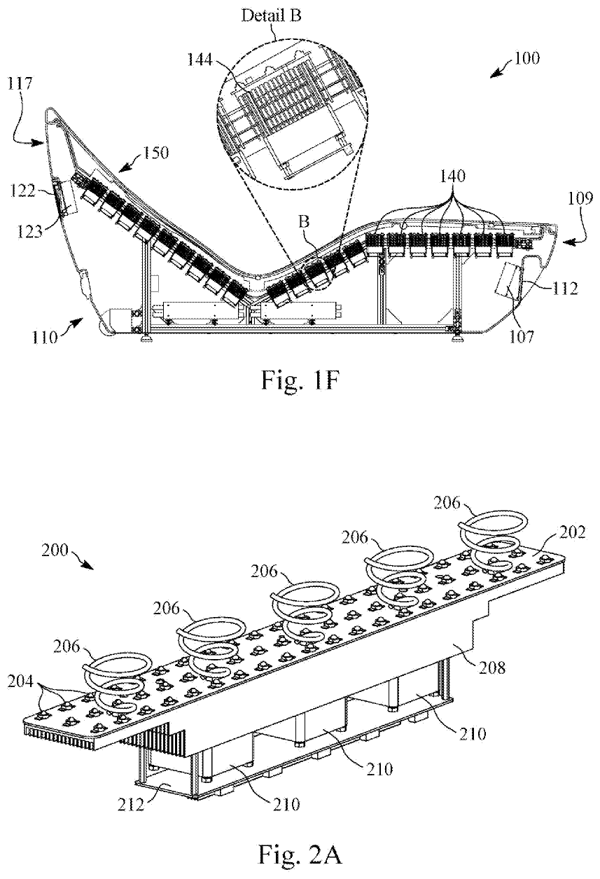

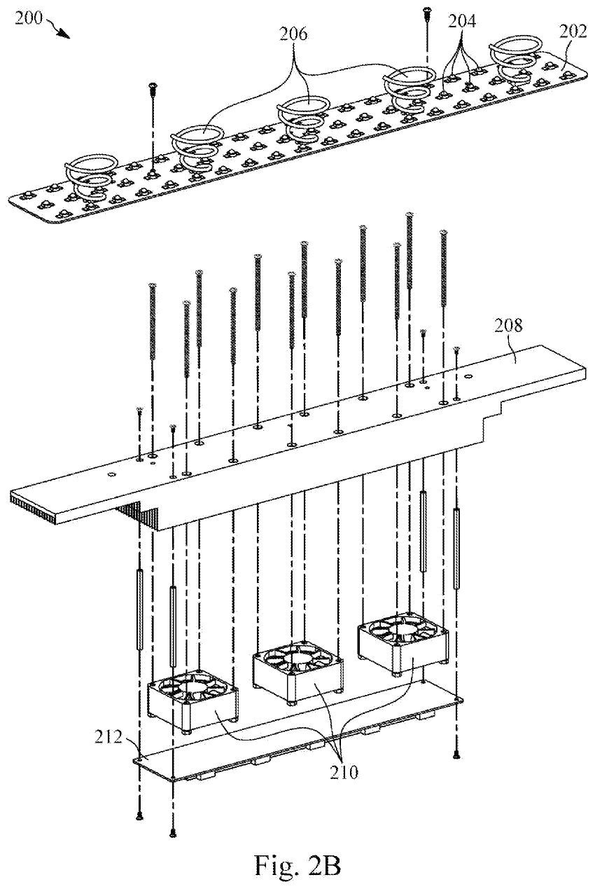

The lounger 100 further includes a user interface 111. The user interface 111 is mechanically coupled to the frame 110. The user interface 111 may include push buttons, capacitive pressure sensors, or resistive pressure sensors. In addition, a display unit (not shown) may also be incorporated into the user interface 111. The user interface 111 is configured to enable the user to manually provide control input signals for several operational modes of the lounger 100. In that regard, the user interface 111 may be configured to receive a control input signal to modify irradiation characteristics of a plurality of irradiation sources (See FIGS. 2A and 2B) of the lounger 100. Furthermore, the lounger 100 also includes a wireless charging pod 113 for wirelessly charging an electronic device such as a smartphone, a power bank, or a wireless Bluetooth headset. In that regard, the wireless charging pod 113 has been provided with a transmitter induction coil (not shown). The wireless charging pod 113 is configured to receive therewithin the electronic device that includes a receiver induction coil (not shown). The receiver induction coil, therefore, is configured to generate an Electro-motive force (EMF) when brought within a time-varying magnetic field generated by the transmitter induction coil.

A plurality of ground-engaging members 114 have been provided at a bottom portion 115 of the frame 110. In several embodiments, the plurality of ground-engaging members 114 may be wheels, slides, rotating belts, etc. The plurality of ground-engaging members 114 are configured to be making contact with a locating surface 135 on which the frame 110 or the lounger 100 be located. In several embodiments of the invention, the plurality of ground-engaging members 114 may be made up of combinations of metallic and polymer materials to provide strength, rigidity, and durability, while also keeping respective weights minimal. The plurality of ground-engaging members 114 are configured to facilitate transportation or movement of the lounger 100 from one location to another. In addition to the ground engaging members 114, the bottom portion 115 also includes a plurality of suction cups 116 configured to affix and stabilize the frame 110 and hence the lounger 100 when located on the locating surface 135. Also, the bottom portion 115 includes a lower cover 119 made up of Aluminum to prevent the frame 110 from ingress of dust and/or water. A power switch 118, a power port 120 configured to receive a power cable (not shown), and a second exhaust vent 122 have been provided in a rear portion 117 of the frame 110.

Referring to FIG. 1E, the frame 110, and hence the lounger 100 have been located on the locating surface 135. Furthermore, the frame 110 includes a peripheral wall 125 with an inner surface 127 and an upper surface 129. An inward protrusion 130 extends internally from the inner surface 127. Furthermore, the inward protrusion 130 has been disposed at a predetermined depth ‘d’ from the upper surface 129. The inward protrusion 130 includes a first protrusion portion 132 parallel to the locating surface 135. Further, the inward protrusion 130 includes a second protrusion portion 134 at a first predetermined angle to the first protrusion portion 132. The second protrusion portion 134 extends rearwardly and downwardly. A third protrusion portion 136 of the inward protrusion 130 is disposed at a second predetermined angle and extends rearwardly and upwardly thereby providing the inward protrusion 130 a lounger-like shape.

The lounger 100 further has been depicted to include a plurality of irradiation boards 140 configured to be located on the inward protrusion 130. The plurality of irradiation boards 140 have been provided with a plurality of irradiation sources 145 with each one of the plurality of irradiation boards 140 including several irradiation sources. In that regard, the plurality of irradiation boards 140 includes at least a first irradiation board 142 configured to be located on the first protrusion portion 132, at least a second irradiation board 144 configured to be located on the second protrusion portion 134, and at least a third irradiation board 146 configured to be located on the third protrusion portion 136. Similarly, the upper cover 150 includes a first cover portion 152 parallel to the first protrusion portion 132, a second cover portion 154 parallel to the second protrusion portion 134, and a third cover portion 156 parallel to the third protrusion portion 136. Referring to FIG. 1F, a detailed view of portion B depicting the second irradiation board 144 has been illustrated. The first exhaust fan 107 is located at the first exhaust vent 112, and a second exhaust fan 123 is located at the second exhaust vent 122. The first 107 and the second 123 exhaust fans are configured to force out the heated air built up within the frame 110 through the first 112 and the second 122 exhaust vents, respectively.

FIG. 2A illustrates a perspective view of an irradiation board 200 of the plurality of irradiation boards 140 of the lounger 100, in accordance with an embodiment of the present invention. FIG. 2B illustrates an exploded view of the irradiation board 200 of FIG. 2A. The irradiation board 200 includes a first Printed Circuit Board (PCB) 202. Furthermore, several irradiation sources 204 are electrically coupled to the first PCB 202. The several irradiation sources 204 may be selected from a group consisting of Light Emitting Diodes (LEDs) and lasers. In addition, one or more pressure sensors 206, acting as multi-point pressure switches corresponding to an equal number of subsets of the several irradiation sources 204, have also been electrically coupled to the first PCB 202. In several embodiments of the invention, the one or more pressure sensors 206 include capacitive antennae. The first PCB 202 may also additionally include control architecture including processors, memory units, communication interfaces, and storage devices. The memory units and/or the storage devices may be provided with machine-readable instructions that when executed by one or more of the processors would enable several operational aspects of the lounger to be automated. Some of those aspects have been discussed in the following discussion.

A heat sink 208 is thermally coupled to the first PCB 202 for absorbing heat generated by electronic components of the first PCB 202. The heat sink 208 is located under the first PCB 202. In several embodiments of the invention, the heat sink 208 is made up of a thermally conducting material, such as aluminum or copper. In addition, the heat sink 208 may be provided with several fins to increase the total surface area exposed to air for enhancing heat lost due to convection. Further, a plurality of cooling fans 210 are located under the heat sink 208. The plurality of cooling fans 210 are further electrically coupled to the first PCB 202 and/or a second PCB 212 and derive electrical power through the first 202 and/or the second 212 PCBs. The electrical power to the first 202 and the second 212 PCBs may be received through an AC or DC power supply delivered via the power port 120.

The plurality of cooling fans 210 are configured to generate air draft towards the heat sink 208 to enhance heat transfer between the heat sink 208 and the air and therefore enhance cooling efficiency of the irradiation board 200. The heated air after absorbing heat from the heat sink 208 is discharged to the atmosphere outside of the frame 110 by the first 107 and the second 123 exhaust fans, through the first 112 and the second 122 exhaust vents. In several embodiments of the invention, the second PCB 212 may be electrically coupled to the first PCB 202, and the plurality of cooling fans 210 may be controlled through the control architecture of the first PCB 202. Alternatively, the second PCB 212 may include a second control architecture that may be in electrical communication with the control architecture of the first PCB 202.

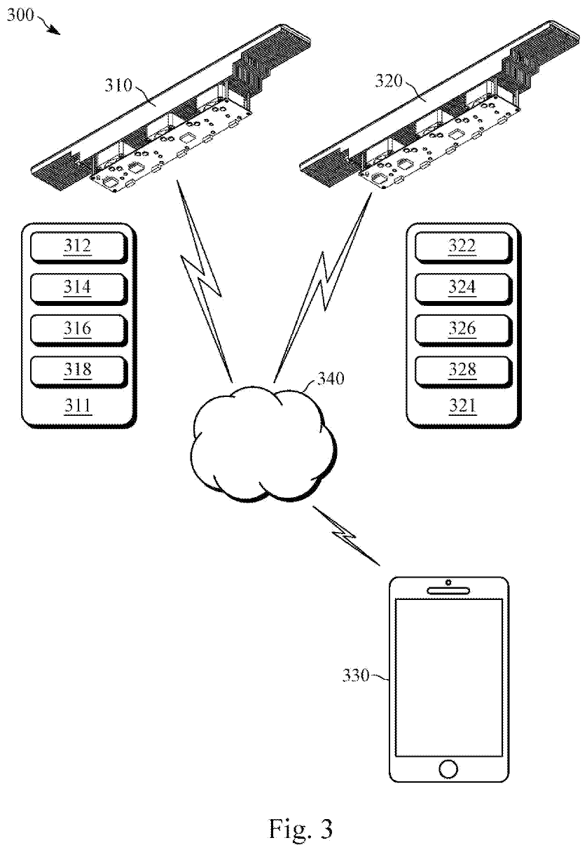

FIG. 3 illustrates an environment 300 depicting two irradiation boards including a first irradiation board 310 and a second irradiation board 320 of the plurality of irradiation boards 140 in communication with a user computing device 330, through a communication network 340, in accordance with an embodiment of the present invention. The user computing device 330 may be a smartphone, a tablet PC, a desktop PC, a notebook PC, and the like. The first irradiation board 310 includes a first control module 311. The first control module 311 includes a first processor 312, a first memory unit 314, a first communication interface 316, and a first storage device 318. Similarly, the second irradiation board 320 includes a second control module 321. The second control module 321 includes a second processor 322, a second memory unit 324, a second communication interface 326, and a second storage device 328. The first irradiation board 310 is in communication with the user computing device 330 through the first communication interface 316 and the communication network 340, and the second irradiation board 320 is in communication with the user computing device 330 through the second communication interface 326 and the communication network 340. In that regard, the lounger 100 is configured to receive a control input signal from the user computing device 330 at any one or more of the plurality of irradiation boards 140. The respective one or more processors of the one or more of the plurality of irradiation boards 140 are enabled to modify irradiation characteristics (such as wavelength, pulse width, mode of operation (pulsed or continuous), time of exposure, intensity, area coverage, number or irradiation sources illuminated) of the plurality of irradiation sources 145 in response to the receipt of the control input signal.

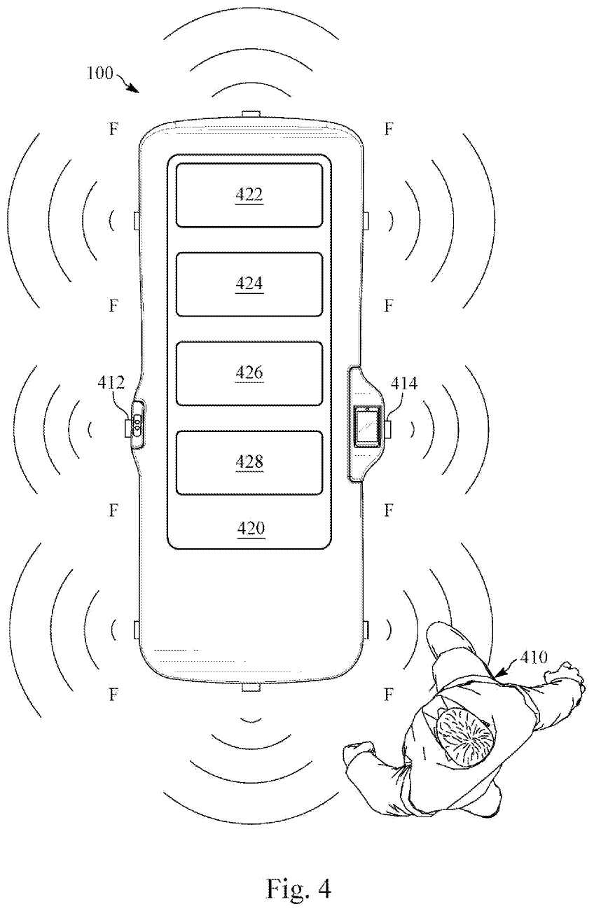

FIG. 4 illustrates a user 410 approaching a detection field F in the vicinity of the lounger 100, in accordance with an embodiment of the present invention. In addition to the pressure sensors, for example the one or more pressure sensors 206, the lounger 100 also includes a plurality of proximity sensors 412 and 414. The plurality of proximity sensors 412 and 414 may be selected from a group consisting of Hall-Effect Sensors and Optical Sensors (including Single Lense Reflectors, CMOS sensors, CCD sensors, IR cameras, etc.). The plurality of proximity sensors 412 and 414 thus define a detection field F in the vicinity of the lounger 100. In the case of the Hall-Effect Sensors, the detection field F will be a magnetic field, whereas, in the case of optical sensors, the detection field F would be an electromagnetic field. The sensing radius, i.e., the largest distance to which the plurality of proximity sensors 412 and 414 can accurately detect the presence of an object may vary as per several factors such as cost, make, quality, sensitivity, model, type, and the like. Furthermore, the lounger 100, in addition to individual control modules of the plurality of irradiation boards 140, also includes a central control module 420. The central control module 420 includes a central processor 422, a central memory unit 424, a central communication interface 426, and a central storage device 428.

As the user 410 approaches the lounger 100, a plurality of sensors including the one or more pressure sensors 206 and the plurality of proximity sensors 412 and 414 transmit input data to the central processor 422. The central processor 422 executing machine-readable instructions stored in the central memory unit 424 receives the input data from the plurality of sensors. The input data is indicative of the presence of the user 410 in a predefined 3-dimensional space (for example, the detection field F) around the frame 110. Furthermore, the central processor 422 determines the location of the user 410 from the input data. Once the location of the user 410 has been identified, the central processor 422 activates one or more irradiation sources that are directed towards the user. It is to be noted that the activated one or more irradiation sources may belong to a single irradiation board or may be distributed amongst several irradiation boards depending upon the location and physiological characteristics of the user 410.

In several embodiments of the invention, using the input data, the central processor 422 may further determine the demographic of the user 410. For example, from image data, the central processor 422 may determine whether the user 410 is a man or a woman, or the central processor 422 may determine whether the user 410 is an adult, a teenager, a pre-teenager, a child, a toddler, or an infant. Or whether the user 410 is an Asian, Hispanic, Caucasian, or African. Alternately, the central processor 422 may determine the species of the user 410. In that regard, the central processor 422, based on pressure data, image data, and/or magnetic field data, may determine whether the user 410 is a human being, a dog, a cat, etc. The determination of the demographic and/or the species to which the user 410 belongs may be performed by the central processor 422 using Artificial Intelligence (AI) developed through Machine Learning and/or Deep Learning algorithms trained on a large amount of historical training data. Once the central processor 422 has identified the demographic and/or the species to which the user 410 belongs, the central processor 422 may then modify the irradiation characteristics of the one or more irradiation sources based on the determined demographic and/or species. For example, the intensity of irradiation and exposure times may be kept minimal for infants, toddlers, and pets. Similarly, different species may exhibit different skin sensitivity to a particular wavelength of the irradiation.

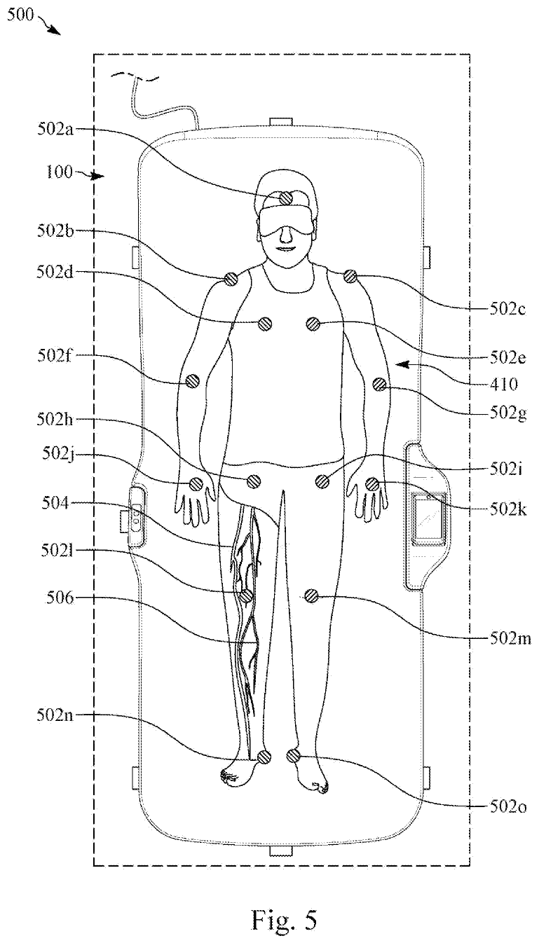

FIG. 5 illustrates a use case scenario 500 of the lounger 100, with the user 410 lying on the lounger 100, in accordance with an embodiment of the present invention. With the user 410 lying on the lounger 100, several pressure sensors 502a, 502b, 502c, 502d, 502e, 502f, 502g, 502h, 502i, 502j, 502k, 502l, 502m, 502n, and 502o may be activated. In such a scenario, the central processor 422 may be able to identify one or more of a predetermined body portion, a predetermined muscle group, and a predetermined group of blood vessels by knowing the locations of all of the pressure sensors 502a, 502b, 502c, 502d, 502e, 502f, 502g, 502h, 502i, 502j, 502k, 502l, 502m, 502n, and 502o. In that regard, a table mapping each of the pressure sensors 502a, 502b, 502c, 502d, 502e, 502f, 502g, 502h, 502i, 502j, 502k, 502l, 502m, 502n, and 502o with their location on the upper cover 150 of the lounger 100 may be maintained in the central memory unit 424 or central storage device 428 and directly accessible to the processor 422.

Moreover, machine-readable instructions in the central memory unit 424 may further include data defining relative pressures applied by different portions of the body of the user 410. Such as the hips and the head of the user 410 are likely to apply a greater amount of pressure when compared to skin under the knees or the elbows. An intermediate amount of pressure may be applied by the ankles of the user 410. Such relative pressure values may be generated by the central processor 422 when trained on historical data using Machine Learning and/or Deep Learning Algorithms. Hence, based on pressure values reported by the pressure sensors 502a, 502b, 502c, 502d, 502e, 502f, 502g, 502h, 502i, 502j, 502k, 502l, 502m, 502n, and 502o, the processor 422 may be able to map the entire body of the user 410. For example, by analyzing the pressure values reported by the pressure sensors 502h, 502l, and 502n, the central processor 422 may be able to identify the deep femoral vein 504 and the great saphenous vein 506 of the user 410. Further, the central processor 422 may then be able to activate the one or more irradiation sources of the plurality of irradiation sources 145 that are directed towards the location of the one or more of the identified predetermined body portion, the predetermined muscle group, and the predetermined group of blood vessels.

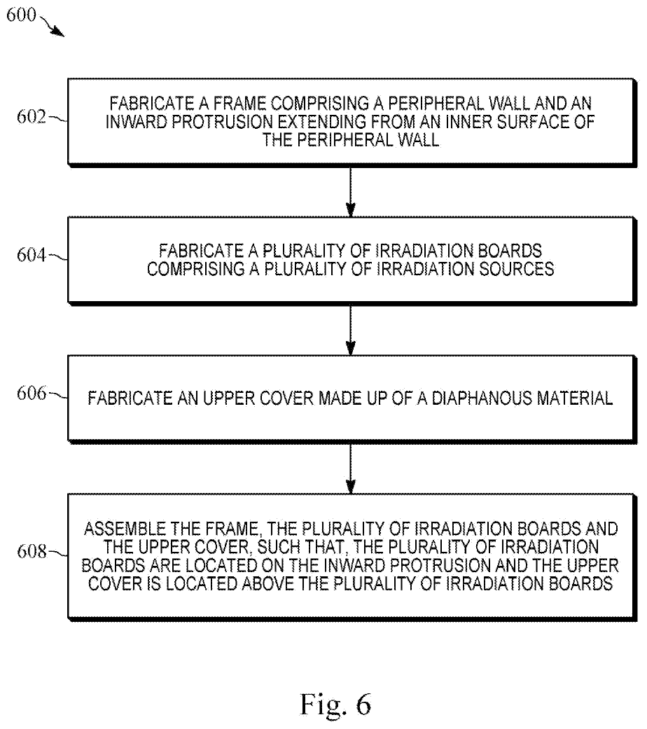

FIG. 6 illustrates a method 600 of manufacturing the lounger 100, in accordance with an embodiment of the present invention. The method 600 begins at Step 602 by fabricating the frame 110 including the peripheral wall 125 and an inward protrusion 130 extending internally from the inner surface 127 of the peripheral wall. The inward protrusion 130 is disposed at the predetermined depth d from the upper surface 129 of the peripheral wall 125. In one embodiment of the invention, the inward protrusion 130 includes the first protrusion portion 132 parallel to the locating surface 135 for locating the frame 110, the second protrusion portion 134 at the first predetermined angle to the first protrusion portion 132 and extending rearwardly and downwardly, and the third protrusion portion 136 at the second predetermined angle to the second protrusion portion 134 and extending rearwardly and upwardly. In several embodiments of the invention, the method further includes providing the user interface 111 configured to receive the control input signal to modify the irradiation characteristics of the plurality of irradiation sources 145. Furthermore, in several embodiments, a wireless charging pod 113 is provided in the frame 110. The wireless charging pod 113 is provided with the transmitter induction coil, the wireless charging pod 113 is configured to receive an electronic device including a receiver induction coil, the receiver induction coil is configured to generate an Electro-motive force (EMF) when brought within a time-varying magnetic field generated by the transmitter induction coil.

At Step 604, the plurality of irradiation boards 140 including the plurality of irradiation sources 145 are fabricated. Further, each irradiation board 200 of the plurality of irradiation boards 140 includes one or more pressure sensors 206 and several irradiation sources 204 electrically coupled to the first Printed Circuit Board (PCB) 202, the heat sink 208 thermally coupled to the first PCB 202 and located under the first PCB 202, and the plurality of cooling fans 210 electrically coupled to the first PCB 202 and/or a second PCB 212, and located under the heat sink 208. In several embodiments of the invention, the one or more exhaust vents 112 and 212 are located in the frame 110 for dissipating heated air generated by the plurality of cooling fans 210.

At Step 606, the upper cover 150 is fabricated from a diaphanous material. The upper cover 150 includes the first cover portion 152 parallel to the first protrusion portion 132, the second cover portion 154 parallel to the second protrusion portion 134, and the third cover portion 156 parallel to the third protrusion portion 136.

At Step 608, the frame 110, the plurality of irradiation boards 140, and the upper cover 150 are assembled, such that, the plurality of irradiation boards 140 are located on the inward protrusion 130 and the upper cover 150 is located above the plurality of irradiation boards 140.

The embodiments of the present invention as discussed above offer several advantages. For instance, the light therapy lounger is simple in construction and cost-effective to manufacture. The design utilizes readily available materials and components. The control architecture is provided both centrally and in a distributed manner with each irradiation board allowing for programming of several different modes of operation. Artificial Intelligence-based monitoring and control of irradiation characteristics allows for the delivery of targeted and user-specific therapy. Construction materials are relatively lighter in weight and when combined with the ground-engaging members provided make the light therapy lounger easily transportable from one place to another. Also, suction cups allow for easy securement and removal from any kind of locating surface.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A light therapy lounger, comprising:
a frame comprising a peripheral wall and an inward protrusion extending internally from an inner surface of the peripheral wall, the inward protrusion disposed at a predetermined depth from an upper surface of the peripheral wall;
a plurality of irradiation boards comprising a plurality of irradiation sources, the plurality of irradiation boards configured to be located on the inward protrusion; and
an upper cover made up of a diaphanous material, the upper cover configured to be located above the plurality of irradiation boards,
wherein each irradiation board of the plurality of irradiation boards comprises:
one or more pressure sensors and several irradiation sources electrically coupled to a first Printed Circuit Board (PCB),
a heat sink thermally coupled to the first PCB and located under the first PCB, and
a plurality of cooling fans electrically coupled to the first PCB and/or a second PCB and located under the heat sink.

2. The light therapy lounger as claimed in claim 1, wherein:
the inward protrusion comprises a first protrusion portion parallel to a locating surface for locating the frame, a second protrusion portion at a first predetermined angle to the first protrusion portion and extending rearwardly and downwardly, and a third protrusion portion at a second predetermined angle to the second protrusion portion and extending rearwardly and upwardly,
the plurality of irradiation boards comprises at least a first irradiation board configured to be located on the first protrusion portion, at least a second irradiation board configured to be located on the second protrusion portion, and at least a third irradiation board configured to be located on the third protrusion portion, and
the upper cover comprises a first cover portion parallel to the first protrusion portion, a second cover portion parallel to the second protrusion portion, and a third cover portion parallel to the third protrusion portion.

3. The light therapy lounger as claimed in claim 1, wherein the plurality of irradiation sources is selected from a group consisting of Light Emitting Diodes (LEDs) and lasers.

4. The light therapy lounger as claimed in claim 1, further comprising one or more exhaust vents located in the frame for dissipating heated air generated by the plurality of cooling fans.

5. The light therapy lounger as claimed in claim 1, further comprising a wireless charging pod provided with a transmitter induction coil, the wireless charging pod configured to receive an electronic device comprising a receiver induction coil, the receiver induction coil configured to generate an Electro-motive force (EMF) when brought within a time-varying magnetic field generated by the transmitter induction coil.

6. The light therapy lounger as claimed in claim 1, further comprising a user interface configured to receive a control input signal to modify irradiation characteristics of the plurality of irradiation sources.

7. The light therapy lounger as claimed in claim 1, wherein the frame further comprises a lower cover made up of aluminum material.

8. The light therapy lounger as claimed in claim 1, further comprising a plurality of sensors, the one or more pressure sensors representing a subset of the plurality of sensors, a processor, and a memory unit, the memory unit comprising machine-readable instructions that when executed by the processor, enable the processor to:
receive input data from the plurality of sensors, the input data indicative of presence of a user within a predefined 3-Dimensional space around the frame;
determine a location of the user using the input data; and
activate one or more irradiation sources of the plurality of irradiation sources directed towards the location of the user.

9. The light therapy lounger as claimed in claim 8, wherein the processor is further enabled to:
determine a demographic and/or a species to which the user belongs, and
modify irradiation characteristics of the one or more irradiation sources based on the determined demographic and/or species.

10. The light therapy lounger as claimed in claim 8, wherein the processor is further enabled to:
identify a location of one or more of a predetermined body portion, a predetermined muscle group, and a predetermined group of blood vessels; and
activate one or more irradiation sources of the plurality of irradiation sources, the activated one or more irradiation sources directed towards the location of the one or more of the identified predetermined body portion, the predetermined muscle group, and the predetermined group of blood vessels.

11. The light therapy lounger as claimed in claim 8, further comprising a communication interface configured to receive a control input signal, from a user computing device, the processor further enabled to modify irradiation characteristics of the plurality of irradiation sources in response to the receipt of the control input signal.

12. The light therapy lounger as claimed in claim 8, wherein the machine-readable instructions comprised in the memory unit correspond to implementation of Artificial Intelligence (AI) developed through Machine Learning and/or Deep Learning algorithms trained on historical training data.

13. A light therapy lounger, comprising:
a frame comprising a peripheral wall and an inward protrusion extending internally from an inner surface of the peripheral wall, the inward protrusion disposed at a predetermined depth from an upper surface of the peripheral wall;
a plurality of irradiation boards comprising a plurality of irradiation sources, the plurality of boards configured to be located on the inward protrusion, wherein each irradiation board of the plurality of irradiation boards comprises one or more pressure sensors and several irradiation sources electrically coupled to a first Printed Circuit Board (PCB), a heat sink thermally coupled to the first PCB and located under the first PCB, and a plurality of cooling fans electrically coupled to the first PCB and/or a second PCB, and located under the heat sink; and
an upper cover made up of a diaphanous material, the upper cover configured to be located above the plurality of irradiation boards;
a plurality of sensors comprising a plurality of proximity sensors and a plurality of pressure sensors;
a memory unit, the memory unit comprising machine-readable instructions that correspond to implementation of Artificial Intelligence (AI) developed through Machine Learning and/or Deep Learning algorithms trained on historical training data,
a processor operably connected to the memory unit, the machine-readable instructions when executed by the processor, enable the processor to perform one or more of:
receive input data from the plurality of sensors, the input data indicative of presence of a user within a predefined 3-Dimensional space around the frame,
determine a location of the user using the input data,
determine a demographic and/or a species to which the user belongs,
identify a location of one or more of a predetermined body portion, a predetermined muscle group, and a predetermined group of blood vessels,
activate one or more of irradiation sources of the plurality of irradiation sources directed towards the location of the one or more of the identified predetermined body portion, the predetermined muscle group, and the predetermined set of blood vessels, and
modify irradiation characteristics of the activated one or more irradiation sources based on the determined demographic and/or the species.

14. The light therapy lounger as claimed in claim 13, further comprising a communication interface configured to receive a control input signal, from a user computing device, the processor further enabled to modify irradiation characteristics of the plurality of irradiation sources in response to the receipt of the control input signal.

15. The light therapy lounger as claimed in claim 13, wherein:
the inward protrusion comprises a first protrusion portion parallel to a locating surface for locating the frame, a second protrusion portion at a first predetermined angle to the first protrusion portion and extending downwardly, and a third protrusion portion at a second predetermined angle to the second protrusion portion and extending upwardly,
the plurality of irradiation boards comprises at least a first irradiation board configured to be located on the first protrusion portion, a second irradiation board configured to be located on the second protrusion portion, and a third irradiation board configured to be located on the third protrusion portion, and
the upper cover comprises a first cover portion parallel to the first protrusion portion, a second cover portion at the first predetermined angle to the first cover portion and extending downwardly, and a third cover portion at the second predetermined angle to the second cover portion and extending upwardly.

16. A method of manufacturing a light therapy lounger, the method comprising:
fabricating a frame comprising a peripheral wall and an inward protrusion extending internally from an inner surface of the peripheral wall, the inward protrusion disposed at a predetermined depth from an upper surface of the peripheral wall;
fabricating a plurality of irradiation boards comprising a plurality of irradiation sources;
fabricating an upper cover made up of a diaphanous material; and
assembling the frame, the plurality of irradiation boards and the upper cover, such that, the plurality of irradiation boards are located on the inward protrusion and the upper cover is located above the plurality of irradiation boards,
wherein each irradiation board of the plurality of irradiation boards comprises:
one or more pressure sensors and several irradiation sources electrically coupled to a first Printed Circuit Board (PCB),
a heat sink thermally coupled to the first PCB and located under the first PCB, and
a plurality of cooling fans electrically coupled to the first PCB and/or a second PCB, and located under the heat sink.

17. The method as claimed in claim 16, wherein:
the inward protrusion comprises a first protrusion portion parallel to a locating surface for locating the frame, a second protrusion portion at a first predetermined angle to the first protrusion portion and extending rearwardly and downwardly, and a third protrusion portion at a second predetermined angle to the second protrusion portion and extending rearwardly and upwardly,
the plurality of irradiation boards comprises at least a first irradiation board configured to be located on the first protrusion portion, at least a second irradiation board configured to be located on the second protrusion portion, and at least a third irradiation board configured to be located on the third protrusion portion, and
the upper cover comprises a first cover portion parallel to the first protrusion portion, a second cover portion parallel to the second protrusion portion, and a third cover portion parallel to the third protrusion portion.

18. The method as claimed in claim 16, further comprising providing one or more exhaust vents located in the frame for dissipating heated air generated by the plurality of cooling fans.

19. The method as claimed in claim 16, further comprising providing a user interface configured to receive a control input signal to modify irradiation characteristics of the plurality of irradiation sources.

20. The method as claimed in claim 16, further comprising providing, in the frame, a wireless charging pod provided with a transmitter induction coil, the wireless charging pod configured to receive an electronic device comprising a receiver induction coil, the receiver induction coil configured to generate an Electro-motive force (EMF) when brought within a time-varying magnetic field generated by the transmitter induction coil.
